(19) United States
(12) Patent Application Publication
(10) Pub. No.: US 2008/0113904 A1
(43) Pub. Date: May 15, 2008

(54) Treatment of Neurological Conditions Using Complement C5a Receptor Modulators

(76) Inventors: Trent Martin Woodruff, Brisbane (AU); Stephen Maxwell Taylor, Bellbird Park (AU)

Correspondence Address: MORRISON & FOERSTER LLP, 12531 HIGH BLUFF DRIVE, SUITE 100, SAN DIEGO, CA, 92130-2040, US

(21) Appl. No.: 10/594,358

(22) PCT Filed: Mar. 21, 2005
(86) PCT No.: PCT/AU05/00403

§ 371 Date: Nov. 26, 2007

(30) Foreign Application Priority Data
Mar. 26, 2004 (AU) 2004901652

(51) Int. Cl. A61K 38/12; A61P 25/28

(52) U.S. Cl. 514/9

(57) ABSTRACT

This invention relates to the treatment of neurological conditions with novel cyclic peptidic and peptidomimetic compounds which have the ability to modulate the activity of C5a receptors. The compounds preferably act as antagonists of the C5a receptor, and are active against C5a receptors on polymorphonuclear leukocytes, monocytes, lymphocytes and/or macrophages. In a preferred form of the invention the neurological conditions are neurodegenerative diseases, neuroimmunological disorders, diseases arising from dysfunction of the blood brain barrier, and stroke.

### FIELD OF THE INVENTION

This invention relates to the treatment of neurological conditions with novel cyclic peptidic and peptidomimetic compounds which have the ability to modulate the activity of C5a receptors. The compounds preferably act as antagonists of the C5a receptor, and are active against C5a receptors on polymorphonuclear leukocytes, monocytes, lymphocytes and/or macrophages. In a preferred form of the invention the neurological conditions are neurodegenerative diseases, neuroimmunological disorders, diseases arising from dysfunction of the blood brain barrier, and stroke.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

G protein-coupled receptors are prevalent throughout the human body, comprising approximately 60% of known cellular receptor types, and mediate signal transduction across the cell membrane for a very wide range of endogenous ligands. They participate in a diverse array of physiological and pathophysiological processes, including, but not limited to those associated with cardiovascular, central and peripheral nervous system, reproductive, metabolic, digestive, immunological, inflammatory, and growth disorders, as well as other cell-regulatory and proliferative disorders. Agents which selectively modulate functions of G protein-coupled receptors have important therapeutic applications. These receptors are becoming increasingly recognised as important drug targets, due to their crucial roles in signal transduction (G protein-coupled Receptors, IBC Biomedical Library Series, 1996).

One of the most intensively studied G protein-coupled receptors is the receptor for C5a. C5a is one of the most potent chemotactic agents known. It has a variety of activities, including
(a) recruiting neutrophils and macrophages to sites of injury,
(b) altering neutrophil and macrophages morphology;
(c) inducing neutrophil degranulation;
(d) increasing calcium mobilisation, vascular permeability (oedema) and neutrophil adhesiveness;
(e) inducing contraction of smooth muscle;
(f) stimulating release of inflammatory mediators, including histamine, TNF-α, IL-1, IL-6, IL-8, prostaglandins, and leukotrienes;
(g) stimulating release of lysosomal enzymes;
(h) promoting formation of oxygen radicals; and
(i) enhancing antibody production

### (Gerard and Gerard, 1994).

Agents which limit the pro-inflammatory actions of C5a have potential for inhibiting both acute and chronic inflammation, and its accompanying pain and tissue damage. Because such compounds act upstream from the various inflammatory mediators referred to above, and inhibit the formation of many of these compounds, they may have a more powerful effect in alleviating or preventing inflammatory symptoms than agents which directly inhibit the activity of these mediators or their receptors.

In our previous application No. PCT/AU98/00490, we described the three-dimensional structure of some analogues of the C-terminus of human C5a, and used this information to design novel compounds which bind to the human C5a receptor (C5aR), behaving as either agonists or antagonists of C5a. It had previously been thought that a putative antagonist might require both a C-terminal arginine and a C-terminal carboxylate for receptor binding and antagonist activity. We showed that in fact a terminal carboxylate group is not generally required either for high affinity binding to C5aR or for antagonist activity. Instead we found that a hitherto unrecognised structural feature, a turn conformation, was the key recognition feature for high affinity binding to the human C5a receptor on neutrophils. As described in our International application PCT/AU01/01427, we used these findings to design constrained structural templates which enable hydrophobic groups to be assembled into a hydrophobic array for interaction with a C5a receptor. The entire disclosures of these specifications are incorporated herein by this reference.

Movement disorders constitute a serious health problem, especially amongst the elderly sector of the population. These movement disorders are often the result of brain lesions or neurodegenerative conditions. Disorders involving the basal ganglia which result in movement disorders include Parkinson's disease, Alzheimer's disease, Huntington's chorea and Wilson's disease. Furthermore, dyskinesias often arise as sequelae of cerebral ischaemia and other neurological disorders.

Neurodegenerative conditions are chronic progressive conditions which are generally associated with an inexorable decline in motor and/or cognitive function. Some, such as Alzheimer's disease, Huntington's disease, fronto-temporal dementia (Pick's disease), dementia with Lewy body formation, Parkinson's disease, prion-associated conditions such as Creutzfeld-Jacob disease and new variant Creutzfeld-Jacob disease, and amyotrophic lateral sclerosis (ALS; also known as Lou Gehrig's disease), are associated with deposition of aggregates of protein in the brain. Others, such as multiple sclerosis, involve autoimmune mechanisms. Brain injury resulting from trauma, infection, inborn errors of metabolism, cerebral hemorrhage or cerebral thrombosis is a very common cause of severe disability, including paralysis and/or cognitive impairment. Motor neuron disease comprises a group of severe disorders of the nervous system, each of which is characterized by progressive degeneration of motor neurons. Motor neuron diseases may affect the upper motor neurons, which lead from the brain to the medulla or to the spinal cord, and/or the lower motor neurons, which lead from the spinal cord to the muscles of the body. Spasms and exaggerated reflexes indicate damage to the upper motor neurons. A progressive atrophy and weakness of muscles which have lost their nerve supply indicate damage to the lower motor neurons. Conditions in this group include amyotrophic lateral sclerosis; progressive bulbar palsy; spinal muscular atrophy, including infantile and juvenile types; Kugelberg-Welander syndrome; Duchenne's paralysis; Werdnig-Hoffmann disease; and benign focal amyotrophy. All of these conditions are extremely distressing, and either treatment options are limited and very costly, or no effective treatment is currently available. Consequently there is an urgent need for improved methods of treatment of these conditions, and especially for more cost-effective treatments.

A class of unusual inherited disorders, the polyglutamine repeat disease family, exhibits a phenomenon called anticipation, in which the parents may exhibit no symptoms, but 50 percent of their children develop the disease. This family includes Huntington's disease and other neurodegenerative diseases, including spinal and bulbar muscular atrophy, several forms of spinocerebellar ataxia, and dentatorubral pallidoluysian atrophy. These conditions are characterized by trinucleotide repeats in specific genes, which encode polyglutamine sequences in the corresponding protein which result in formation of insoluble aggregates; however, the causative proteins are otherwise unrelated, and may have no known function. The mechanism whereby the protein aggregates induce the pathology is still unclear.

The symptoms of Huntington's disease include chorea (jerky movements of the arms and legs resulting from loss of motor coordination), difficulties with speech, swallowing, concentration and memory, and psychiatric symptoms such as depression. The age of onset decreases, while the severity of the disease increases, with each subsequent generation.

The characteristic protein, huntingtin, of Huntington's disease has no known function. The first exon of the gene encoding huntingtin contains a series of trincleotide CAG (glutamine) repeats which expands spontaneously between generations. If the expansion exceeds a critical threshold of 35 repeats, it disrupts the normal function of the huntingtin protein. The resulting neurotoxic effects kill the neurons of the cerebral cortex and striatum, with catastrophic effects on memory, higher cognitive functions, and motor coordination. Characteristic striatal lesions are observed.

All humans carry the gene, but if the trinucleotide repeat number is between 5 and 35, there is no effect; however, beyond this figure, the gene becomes unstable, and expands spontaneously. The repeat number correlates strongly with the age of onset; at 200 repeats, children as young as 1 or 2 may begin to exhibit symptoms, and these will die in early childhood. In contrast to this, individuals with 35 to 39 repeats do not always develop the disorder, and those who do tend to develop the disease very late in life. The disorder also appears to develop earlier and becomes more rapidly severe if the defective gene is paternally inherited.

Kennedy disease (spinal and bulbar muscular atrophy), which affects approximately 1 in 50,000 men, is another member of this family of diseases, and is caused by a mutation resulting in a poly-glutamine repeat or expansion in the androgen receptor protein (La Spada, 1991). The disease, which in humans is X-linked, generally develops in men in their 40s, and leads to loss of motor neurones, muscular atrophy and testicular pathology. It has been suggested that testosterone, which binds to the androgen receptor, may play an important role in controlling the disease progression. Females have significantly less circulating testosterone, and this may influence the rate of neurodegeneration.

The four classic symptoms of Parkinson's disease are tremor, rigidity, akinesia and postural changes. Parkinson's disease is also commonly associated with depression, dementia and overall cognitive decline. Parkinson's disease has a prevalence of 1 per 1,000 of the total population, and increasing to 1 per 100 for those aged over 60 years. Degeneration of dopaminergic neurones in the substantia nigra and the subsequent reductions in interstitial concentrations of dopamine in the striatum are critical to the development of this condition; about 80% of cells from the substantia nigra need to be destroyed before the clinical symptoms are manifested.

Current strategies for the treatment of Parkinson's disease are based on transmitter replacement therapy (L-dihydroxyphenylacetic acid (L-DOPA)), inhibition of monoamine oxidase (e.g. Deprenyle), dopamine receptor agonists (e.g. bromocriptine and apomorphine) and anticholinergics (e.g. benztrophine and orphenadrine). However, these treatments, in particular transmitter replacement therapy, do not provide consistent clinical benefit. After prolonged transmitter replacement therapy “on-off” symptoms develop, and this treatment has also been associated with involuntary movements of athetosis and chorea, nausea and vomiting. Moreover, current therapies do not treat the underlying neurodegenerative disorder, so that despite treatment patients show a continuing cognitive decline.

Despite recent progress in the art, there is still a great need for improved therapies for movement disorders, especially Huntington's disease and Parkinson's disease, and neurodegenerative conditions such as ALS. In particular, effective treatments which require less frequent dosing, are associated with fewer and/or less severe side-effects, and/or which control or reverse the underlying neurodegenerative disorder, are required.

Although it is widely thought that inflammatory mechanisms may be involved in the pathogenesis of many neurodegenerative conditions, including Huntington's disease, fronto-temporal dementia (Pick's disease), multiple sclerosis, Alzheimer's disease, Parkinson's disease and aging-associated neurodegeneration (dementia), as well as in acute brain injury caused by stroke or trauma, relatively little attention has been paid to the possible role of complement in these conditions. Complement activation in Huntington's disease, fronto-temporal dementia (Pick's disease) and Alzheimer's disease has been investigated, and it is known that complement components are widely expressed in both normal and pathological brain. Upregulation of several components of the complement pathway, including the complement activators C1r, C4 and C3, the complement regulators C1 inhibitor, clusterin, MCP, DAF and CD59, and the receptors for the anaphylotoxins C3a and C5a, has been detected in brains of Huntington's disease patients. It was suggested that the mutant huntingtin which accumulates in the neurons of the caudate in Huntington's disease might bind to Clq, and thereby initiate activation of the classical complement pathway (Singhrao et al, 1999).

However, there has hitherto been no evidence to suggest that an inhibitor of the C5a receptor, and in particular a low molecular weight antagonist of this receptor, might be useful in the treatment of neurological or neurodegenerative conditions involving inflammation.

### SUMMARY OF THE INVENTION

We have now surprisingly found that a low molecular weight antagonist of the C5a receptor, a small cyclic peptide, is able to prevent or alleviate the neurological damage in an animal model of metabolic-ischemia-induced neuronal cell death. This animal model is used as a model system for a variety of neurological conditions, especially conditions associated with lesions of the striatum. We have also demonstrated improved survival and delayed loss of motor function in an animal model of ALS.

According to a first aspect, the invention provides a method of treatment of a neurological or neurodegenerative condition involving inflammation, comprising the step of administering an effective amount of an inhibitor of the C5a receptor to a subject in need of such treatment. Preferably the condition is one associated with increased activity of the complement pathway.

Preferably the inhibitor is a compound which
(a) is an antagonist of the C5a receptor,
(b) has substantially no agonist activity, and
(c) is a cyclic peptide or peptidomimetic compound of Formula I

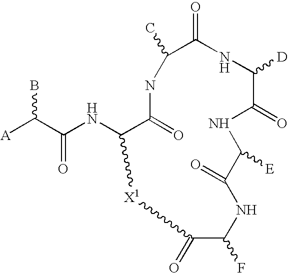

where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl.

B is an alkyl, aryl, phenyl, benzyl, naphthyl or indole group, or the side chain of a D- or L-amino acid such as L-phenylalanine or L-phenylglycine, but is not the side chain of glycine, D-phenylalanine, L-homophenylalanine, L-tryptophan, L-homotryptophan, L-tyrosine, or L-homotyrosine;

C is a small substituent, such as the side chain of a D-, L- or homo-amino acid such as glycine, alanine, leucine, valine, proline, hydroxyproline, or thioproline, but is preferably not a bulky substituent such as isoleucine, phenylalanine, or cyclohexylalanine;

D is the side chain of a neutral D-amino acid such as D-Leucine, D-homoleucine, D-cyclohexylalanine, D-homocyclohexylalanine, D-valine, D-norleucine, D-homo-norleucine, D-phenylalanine, D-tetrahydroisoquinoline, D-glutamine, D-glutamate, or D-tyrosine, but is preferably not a small substituent such as the side chain of glycine or D-alanine, a bulky planar side chain such as D-tryptophan, or a bulky charged side chain such as D-arginine or D-Lysine;

E is a bulky substituent, such as the side chain of an amino acid selected from the group consisting of L-phenylalanine, L-tryptophan and L-homotryptophan, or is L-1-napthyl or L-3-benzothienyl alanine, but is not the side chain of D-tryptophan, L-N-methyltryptophan, L-homophenylalanine, L-2-naphthyl L-tetrahydroisoquinoline, L-cyclohexylalanine, D-leucine, L-fluorenylalanine, or L-histidine;

F is the side chain of L-arginine, L-homoarginine, L-citrulline, or L-canavanine, or a bioisostere thereof, i.e. a side chain in which the terminal guanidine or urea group is retained, but the carbon backbone is replaced by a group which has different structure but is such that the side chain as a whole reacts with the target protein in the same way as the parent group; and

X is —(CH₂)ₙNH— or (CH₂)ₙ—S—, where n is an integer of from 1 to 4, preferably 2 or 3; —(CH₂)₂O—; —(CH₂)₃O—; —(CH₂)₃—; —(CH₂)₄—; —CH₂COCHRNH—; or —CH₂—CHCOCHRNH—, where R is the side chain of any common or uncommon amino acid.

In C, both the cis and trans forms of hydroxyproline and thioproline may be used.

Preferably A is an acetamide group, an aminomethyl group, or a substituted or unsubstituted sulphonamide group.

Preferably where A is a substituted sulphonamide, the substituent is an alkyl chain of 1 to 6, preferably 1 to 4 carbon atoms, or a phenyl or toluoyl group.

In a particularly preferred embodiment, the compound has antagonist activity against C5aR, and has substantially no C5a agonist activity.

The compound is preferably an antagonist of C5a receptors on human and mammalian cells including, but not limited to, human polymorphonuclear leukocytes, monocytes, lymphocytes and macrophages. The compound preferably binds potently and selectively to C5a receptors, and more preferably has potent antagonist activity at sub-micromolar concentrations. Even more preferably the compound has a receptor affinity IC50<25 μM, and an antagonist potency IC50<1 μM.

Most preferably the compound is compound 1 (PMX53; AcF[OP-DCha-WR]), compound 33 (PMX273; AcF[OP-DPhe-WR]), compound 60 (PMX95; AcF[OP-DCha-FR]) or compound 45 (PMX201; AcF[OP-DCha-WCit]) described in International Patent Application No. PCT/AU02/01427, or HC-[OPdChaWR] (PMX205) or HC—[OPdPheWR] (PMX218). The structures of these cyclic peptides are illustrated in FIG. 1.

In one preferred embodiment the compound is able to cross the blood-brain barrier. In a particularly preferred embodiment the compound is PMX205 or PMX53.

In one preferred form of the invention the condition is a neurodegenerative condition associated with striatal lesions and/or polyglutamine repeats.

In this form of the invention the condition is more preferably selected from the group consisting of Huntington's disease, spinal and bulbar muscular atrophy, spinocerebellar ataxia, dentatorubral pallidoluysian atrophy, striatal injury, and acute striatal necrosis associated with Type I glutaric aciduria.

In another preferred form of the invention the condition is a motor neuron disease such as amyotrophic lateral sclerosis; progressive bulbar palsy; spinal muscular atrophy, including infantile and juvenile types; Kugelberg-Welander syndrome; Duchenne's paralysis; Werdnig-Hoffmann disease; and benign focal amyotrophy.

In a third preferred form of the invention the condition is a disorder involving neurodegeneration and/or ischemic damage, including but not limited to Parkinson's disease, Alzheimer's disease, Wilson's disease, and pathologies arising as sequelae of cerebral ischaemia and other neurological disorders, including diseases associated with dysfunction of the blood-brain barrier. It is known that the striatal region is the area of the brain most commonly affected by stroke, and the 3-NP model is a useful model of stroke, because 3-NP induces brain anoxia. Parkinson's-type disorders of particular interest are Parkinson's disease, drug-induced Parkinsonism, post-encephalitic Parkinsonism, Parkinsonism induced by poisoning (for example MPTP, manganese or carbon monoxide) and post-traumatic Parkinson's disease (punch-drunk syndrome). Other movement disorders in which the therapy may be of benefit include progressive supranuclear palsy, Huntington's disease, multiple system atrophy, corticobasal degeneration, Wilson's disease, Hallervorden-Spatz disease (neurodegeneration with brain iron accumulation), progressive pallidal atrophy, Dopa-responsive dystonia-Parkinsonism, spasticity, Alzheimer's disease and other disorders of the basal ganglia which result in abnormal movement or posture.

The inhibitor may be used in conjunction with one or more other agents for the treatment of these conditions. For example, various agents including trehalose, copaxone, short single-stranded oligonucleotides, creatine, minocycline and histone deacetylase inhibitors have been suggested for the treatment of Huntington's disease. Riluzone, a glutamate pathway antagonist, is approved for the treatment of ALS, and creatine, recombinant human IGF-1 and ciliary neurotrophic factor are all in clinical trial for this condition. It has recently been suggested that the familial form of ALS could be treated by suppression of the abnormal SOD1 gene using RNA interference coupled with gene therapy to introduce a normal SOD1 gene (Raoul et al., 2005; Ralph et al., 2005). However, it is likely that this approach will take many years to reach the clinic. The anti-androgen agent leuprorelin is being tested for treatment of spinal and bulbar muscular atrophy (Katsuno et al, 2003). The transmitter replacement L-dihydroxyphenylacetic acid (L-DOPA), monoamine oxidase inhibitors such as Deprenyl®, dopamine receptor agonists such as bromocriptine and apomorphine and anticholinergics such as benztrophine and orphenadrine are currently used in the treatment of Parkinson's disease.

The compositions of the invention may be formulated for oral, parenteral, inhalational, intranasal, rectal or transdermal use, but oral or parenteral formulations are preferred. It is expected that most if not all compounds of the invention will be stable in the presence of metabolic enzymes, such as those of the gut, blood, lung or intracellular enzymes. Such stability can readily be tested by routine methods known to those skilled in the art.

Optionally the formulation may include an agent or carrier which promotes transfer of the compound across the blood-brain barrier. Several such agents are known in the art, for instance osmotically active agents such as mannitol.

Suitable formulations for administration by any desired route may be prepared by standard methods, for example by reference to well-known textbooks such as Remington: The Science and Practice of Pharmacy, Vol. II, 2000 (20^{th} edition), A. R. Gennaro (ed), Williams & Wilkins, Pennsylvania.

While the invention is not in any way restricted to the treatment of any particular animal or species, it is particularly contemplated that the method of the invention will be useful in medical treatment of humans, and will also be useful in veterinary treatment, particularly of companion animals such as cats and dogs, livestock such as cattle, horses and sheep, and zoo animals, including non-human primates, large bovids, felids, ungulates and canids.

The compound may be administered at any suitable dose and by any suitable route. Oral, transdermal or intranasal administration is preferred, because of the greater convenience and acceptability of these routes. The effective dose will depend on the nature of the condition to be treated, and the age, weight, and underlying state of health of the individual treatment. This will be at the discretion of the attending physician or veterinarian. Suitable dosage levels may readily be determined by trial and error experimentation, using methods which are well known in the art.

### BRIEF DESCRIPTION OF THE FIGURES

FIG. 1 shows the structures of preferred compounds of the invention.

FIG. 2 shows the results of a pilot study of the effect of PMX53 on the 3-NP rat model.

A Weight change;

B Food consumption;

C Neurological/behavioural score;

D Representative cresyl violet-stained sections of the striatum of sham operated (A), untreated (B) and PMX53-treated (C) rats illustrating the degree of necrosis in this region.

FIG. 3 shows the results of an experiment in which PMX53 or comparator drugs were administered by oral gavage.

A Weight change at day 7;

B Food consumption at day 7;

C Neurological/behavioural score;

D Representative cresyl violet-stained sections of the striatum of sham operated (A), untreated (B), PMX53-treated (C) and PMX205-treated (D) rats at day 7 illustrating the degree of necrosis in this region.

FIG. 4 shows the results obtained in the extended study reported in Example 2.

A Weight change at day 7;

B Food consumption at day 7;

C Neurological/behavioural score;

D Lesion size as determined by analysis of Nissl-stained sections of rat brain.

FIG. 5 shows the results of histological and histochemical examinations of sections of rat brain from Example 2.

(a) haematoxylin and eosin stain

(b) naphthyl esterase stain

(c) TUNEL staining for detection of apoptosis.

FIG. 6 shows the results of immunohistochemical examination of sections of rat brain from Example 2.

FIG. 7 shows the effects of treatment with compounds of the invention in a transgenic rat model of ALS.
(a) Time of onset of loss of motor function
(b) Percent survival
(c) Percentage of rats in each group showing onset of motor symptoms over time
(d) Delay between first loss of body weight and onset of motor symptoms.

FIG. 8 shows the levels of compounds of the invention in the brain following i.v. injection.

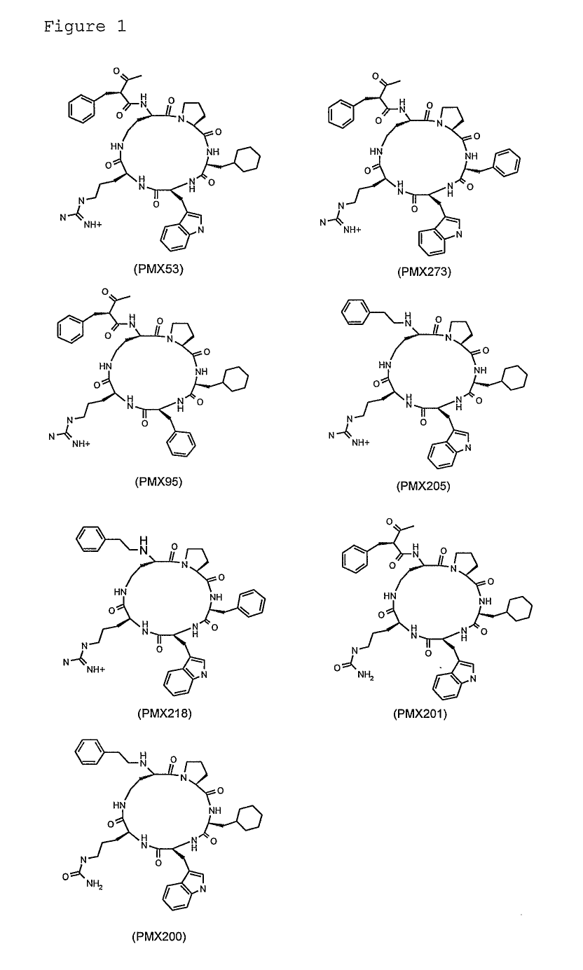

### DETAILED DESCRIPTION OF THE INVENTION

The invention will now be described by way of reference only to the following general methods and experimental examples.

For the purposes of this specification it will be clearly understood that the word “comprising” means “including but not limited to”, and that the word “comprises” has a corresponding meaning.

As used herein, the singular forms “a”, “an”, and “the” include plural reference unless the context clearly dictates otherwise. Thus, for example, a reference to “an enzyme” includes a plurality of such enzymes, and a reference to “an amino acid” is a reference to one or more amino acids.

Where a range of values is expressed, it will be clearly understood that this range encompasses the upper and lower limits of the range, and all values in between these limits.

Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are now described.

Abbreviations used herein are as follows:

### C5aR C5a receptor

### Cit citrulline

dCha D-cyclohexylamine

### DPhe D-phenylalanine

### IL-6 interleukin-6

ip intraperitoneal
i.v. intravenous

### LPS lipopolysaccharide

### MPO myeloperoxidase

### 3-NP 3-nitropropionic acid

### PBS phosphate-buffered saline

### PMN polymorphonuclear granulocyte

### PMSF phenylmethylsulfonyl fluoride

po per os
sc subcutaneous

### TdT Terminal deoxynucleotidyl transferase

### TNF-α tumour necrosis factor-α

Throughout the specification conventional single letter and three-letter codes are used to represent amino acids.

For the purposes of this specification, the term “alkyl” is to be taken to mean a straight, branched, or cyclic, substituted or unsubstituted alkyl chain of 1 to 6, preferably 1 to 4 carbons. Most preferably the alkyl group is a methyl group. The term “acyl” is to be taken to mean a substituted or unsubstituted acyl of 1 to 6, preferably 1 to 4 carbon atoms. Most preferably the acyl group is acetyl. The term “aryl” is to be understood to mean a substituted or unsubstituted homocyclic or heterocyclic aryl group, in which the ring preferably has 5 or 6 members.

A “common” amino acid is a L-amino acid selected from the group consisting of glycine, leucine, isoleucine, valine, alanine, phenylalanine, tyrosine, tryptophan, aspartate, asparagine, glutamate, glutamine, cysteine, methionine, arginine, lysine, proline, serine, threonine and histidine.

An “uncommon” amino acid includes, but is not restricted to, D-amino acids, homo-amino acids, N-alkyl amino acids, dehydroamino acids, aromatic amino acids other than phenylalanine, tyrosine and tryptophan, ortho-, meta- or para-aminobenzoic acid, ornithine, citrulline, canavanine, norleucine, γ-glutamic acid, aminobutyric acid, L-fluorenylalanine, L-3-benzothienylalanine, and α, α-disubstituted amino acids.

Generally, the terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease.

“Treating” as used herein covers any treatment of, or prevention of disease in a vertebrate, a mammal, particularly a human, and includes: preventing the disease from occurring in a subject who may be predisposed to the disease, but has not yet been diagnosed as having it; inhibiting the disease, i.e., arresting its development; or relieving or ameliorating the effects of the disease, i.e., cause regression of the effects of the disease.

The invention includes the use of various pharmaceutical-compositions useful for ameliorating disease. The pharmaceutical compositions according to one embodiment of the invention are prepared by bringing a compound of formula I, analogue, derivatives or salts thereof and one or more pharmaceutically-active agents or combinations of compound of formula I and one or more pharmaceutically-active agents into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg. in Langer, Science, 249: 1527, (1990). Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be

(a) a naturally occurring phosphatide such as lecithin;

(b) a condensation product of an alkylene oxide with a fatty acid, for example, polyoxyethylene stearate;

(c) a condensation product of ethylene oxide with a long chain aliphatic alcohol, for example, heptadecaethylenoxycetanol;

(d) a condensation product of ethylene oxide with a partial ester derived from a fatty acid and hexitol such as polyoxyethylene sorbitol monooleate, or

(e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

Compounds of formula I may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the compound of formula I of the present invention will usually be of the order of about 0.5 mg to about 20 mg per kilogram body weight, with a preferred dosage range between about 0.5 mg to about 10 mg per kilogram body weight per day (from about 0.5 g to about 3 g per patient per day). The amount of active ingredient which may be combined with the carrier materials to produce a single dosage will vary, depending upon the host to be treated and the particular mode of administration. For example, a formulation intended for oral administration to humans may contain about 5 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other therapeutic compounds to provide an operative combination. It is intended to include any chemically compatible combination of pharmaceutically-active agents, as long as the combination does not eliminate the activity of the compound of this invention.

### General Methods

### Peptide Synthesis

Cyclic peptide compounds of formula I are prepared according to methods described in detail in our earlier applications No. PCT/AU98/00490 and No. PCT/AU02/01427, the entire disclosures of which are incorporated herein by this reference. While the invention is specifically illustrated with reference to the compound AcF-[OPdChaWR] (PMX53), whose corresponding linear peptide is Ac-Phe-Orn-Pro-dCha-Trp-Arg, it will be clearly understood that the invention is not limited to this compound.

Compounds 1-6, 17, 20, 28, 30, 31, 36 and 44 disclosed in International patent application No. PCT/AU98/00490 and compounds 10-12, 14, 15, 25, 33, 35, 40, 45, 48, 52, 58, 60, 66, and 68-70 disclosed for the first time in International patent application PCT/AU02/01427 have appreciable antagonist potency (IC50<1 μM) against the C5a receptor on human neutrophils. PMX205, PMX53, and PMX273, PMX201 and PMX218 are most preferred.

We have found that all of the compounds of formula I which have so far been tested have broadly similar pharmacological activities, although the physicochemical properties, potency, and bioavailability of the individual compounds varies somewhat, depending on the specific-substituents.

The general tests described below may be used for initial screening of candidate inhibitor of G protein-coupled receptors, and especially of C5a receptors.

### Drug Preparation and Formulation

The human C5a receptor antagonist AcF-[OPdChaWR] (AcPhe[Orn-Pro-D-Cyclohexylalanine-Trp-Arg]) was synthesized as described above, purified by reversed phase HPLC, and fully characterized by mass spectrometry and proton NMR spectroscopy. The CSa antagonist was prepared in distilled water for oral dosing.

### Receptor-Binding Assay

Assays are performed with fresh human PMNs, isolated as previously described (Sanderson et al, 1995), using a buffer of 50 mM HEPES, 1 mM CaCl₂, 5 mM MgCl₂, 0.5% bovine serum albumin, 0.1% bacitracin and 100 μM phenylmethylsulfonyl fluoride (PMSF). In assays performed at 4° C., buffer, unlabelled human recombinant C5a (Sigma) or test peptide, labelled ¹²⁵I-C5a (˜20 pM) (New England Nuclear, Mass.) prepared by the Hunter/Bolton method and PMNs (0.2×10⁶) are added sequentially to a Millipore Multiscreen assay plate (HV 0.45) having a final volume of 200 μL/well. After incubation for 60 min at 4° C., the samples are filtered and the plate washed once with buffer. Filters are dried, punched and counted in an LKB gamma counter. Non-specific binding is assessed by the inclusion of 1 mM peptide or 100 nM C5a, which typically results in 10-15% total binding.

Data are analysed using non-linear regression and statistics with Dunnett post-test.

### Myeloperoxidase Release Assay for Antagonist Activity

Cells are isolated as previously described (Sanderson et al, 1995) and incubated with cytochalasin B (5 μg/mL, 15 min, 37° C.). Hank's Balanced Salt solution containing 0.15% gelatin and test peptide is added on to a 96 well plate (total volume 100 μL/well), followed by 25 μL cells (4×10⁶/mL). To assess the capacity of each peptide to antagonise C5a, cells are incubated for 5 min at 37° C. with each peptide, followed by addition of C5a (100 nM) and further incubation for 5 min. Then 50 μL of sodium phosphate (0.1M, pH 6.8) is added to each well, the plate was cooled to room temperature, and 25 μL of a fresh mixture of equal volumes of dimethoxybenzidine (5.7 mg/mL) and H₂O₂ (0.51%) is added to each well. The reaction is stopped at 10 min by addition of 2% sodium azide. Absorbances are measured at 450 nm in a Bioscan 450 plate reader, corrected for control values (no peptide), and analysed by non-linear regression.

### Animal and Cellular Models

It is well established that 3-nitropropionic acid (3-NP), an inhibitor of the enzyme succinate dehydrogenase, induces the motor, lesional and/or cognitive effects in rodents and primates which are characteristic of Huntington's disease (Brouillet et al, 1999; Palfi et al, 1996; Blum et al, 2001). 3-NP induces neuronal death selectively in the striatal region, by creating metabolic-induced anoxia. This model has been used in the evaluation of candidate drugs for treatment of Huntington's disease and other conditions. Because selective striatal lesions are induced by 3-NP, this system has also been suggested as a model for the acute striatal necrosis which is common in infants suffering from Type I glutaric aciduria, an inborn error of organic acid metabolism (Strauss and Morton, 2003). Because of the effects of 3-NP on the blood-brain barrier, glutamatergic excitotoxicity, glutamate transport and dopaminergic toxicity, it has also been suggested that this is a useful model for investigation of stroke, dysfunction of the blood-brain barrier, neurodegenerative or neuroimmunological disorders, and neuronal/glial cell death (Nishino et al, 2000). Expression of C3/C4 receptor has been detected in striatal lesions in this model (Shimano et al, 1995).

Transgenic rats expressing SOD1 G93A, a mutant superoxide dismutase 1 (SOD 1), are a well-recognized model for ALS which is widely used for testing of candidate therapeutic compounds. Transgenic rats expressing SOD1 G93A develop hind limb weakness at about 115 days. The pathology seen in these animals is similar to that observed in mutant SOD1 mouse models, with a very marked loss of the astroglial glutamate transporter EAAT2 at end-stage disease, supporting a role for EAAT2 dysfunction in the aetiology of ALS. This transporter is the primary means of maintaining low extracellular glutamate levels, and its loss results in increased extracellular glutamate, leading to excitotoxic degeneration of motor neurons. The earliest changes in EAAT2 expression are detected prior to motor neuron loss (Howland et al, 2002).

Two transgenic mouse models for Huntington's disease are available, one with a knock-in 115-trinucleotide repeat of the human huntingtin gene (Mangiarini et al, 1996), the other with a knock-in copy of the critical first exon of the human gene within its own huntingtin gene.

A cellular model in which Neuro2a cells express a truncated N-terminal huntingtin, containing 60 or 150 glutamines fused to an enhanced green fluorescence protein, has also been described (Wang et al, 1999).

A mouse model for Kennedy disease (spinal and bulbar muscular atrophy) which displays most of the symptoms of the human disease, including muscle weakness and infertility, has recently been developed (McManamny et al (2002).

A mouse model for spinocerebellar ataxia-1 has been described (Klement, I. A. et al. Cell 95, 41-53 (1998).

### General Experimental Protocol

Unless otherwise stated, the protocol for induction of 3-NP neurotoxicity employed in the present study was similar to that of Blum et al (2001; 2002), except that we used 42 mg/kg/day for 7 days rather than 56 mg/kg for 5 days. Briefly, a 90 mg/mL solution of 3-NP in PBS (0.1 M, pH 7.4) was prepared and adjusted to pH 7.4 with 5M NaOH. Alzet osmotic mini-pumps (model 2ML1, delivering 10 μL/hr for 7 days) filled with this solution were implanted into 12 week old male Lewis rats, so that each rat received exactly 42 mg/kg/day.

Rats were housed in individual cages. The rats were anaesthetised with ketamine, xylazine and zolazapam. A pump was inserted s.c. into the back of each rat via an incision between the scapulae, and the incision closed with wound clips. Following recovery from anaesthesia, food intake, body weight and neurological were evaluated daily over the next 7 days. Neurological and behavioural evaluation according to standard criteria was also performed at several points by an observer blinded to the identity of the groups.

Rats were divided into the following groups:

| | |
|---|---|
| Test | Treated with test agent administered in |
| | the drinking water and s.c injection, |
| | or by oral gavage, from 2 days prior to |
| | 3-NP administration (Day-2) until the |
| | end of the experiment |
| Sham-operated | Sham-operated control; no 3-NP |
| Untreated | Given 3-NP but no other treatment |
| Comparative | Given infliximab (5 mg/kg single i.v. |
| | injection on Day 0) or ibuprofen (30 mg/kg |
| | in drinking water |

### Scoring was as Follows

### Dystonia:

| | |
|---|---|
| No Dystonia | 0 |
| Intermittent Dystonia of 1 hindlimb | 1 |
| Intermittent Dystonia of 2 hindlimbs | 2 |
| Permanent Dystonia of hindlimbs | 3 |

### Gait:

| | |
|---|---|
| Normal Gait | 0 |
| Uncoordinated gait and wobbling | 1 |

### Recumbency:

| | |
|---|---|
| Mild Recumbency | 1 |
| (animals lying on one side but showing | |
| uncoordinated movements when stimulated) | |
| Near death recumbency | 2 |

### Cage Grasp:

| | |
|---|---|
| Able to grasp cage with forepaws | 0 |
| Unable to grasp cage with forepaws | 1 |

### Raised Platform Test

| | |
|---|---|
| Able to stay on platform for 10 secs | 0 |
| Unable to stay on platform for 10 secs | 1 |
| TOTAL SCORE: | 8 |

After completion of the study, rats were deeply anaesthetised with xylazine and ketamine. The rats were then perfused with approximately 100 mL of sodium nitrite solution to remove blood, followed by approximately 400 mL of formaldehyde solution to fix the brains in situ. Brains were then carefully removed and stored in formaldehyde solution for at least 3 days, before processing for histology. Slides were stained with cresyl violet and examined by light microscopy.

### EXAMPLE 1

### Pilot Study on Effect of PMX53

A preliminary experiment had shown that a dose of 42 mg/kg/day for 7 days gave reproducible induction of the model. A small pilot study was performed in order to test the effect of PMX53 in the model system. In this study rats treated with PMX53 were compared with sham and untreated controls. A total of 12 rats was used, as follows:

| | |
|---|---|
| Sham | 2 |
| Untreated | 5 |
| PMX53 | 5 |

PMX53 was administered daily at a dose of 2 mg/kg in the drinking water, beginning 2 days prior to 3-NP administration. These PMX53-treated animals were also given a s.c. dose of 1 mg/kg on Days 0, 3, 6 and 8 because they were not eating, and therefore it was thought that they might not have been drinking the water. In subsequent experiments animals were dosed daily by gavage, beginning on Day -2, in order to avoid this potential confounding factor. In this initial experiment, the pumps were removed after 7 days and the skin sutured under light halothane anaesthesia, and the rats were examined daily for another 7 days.

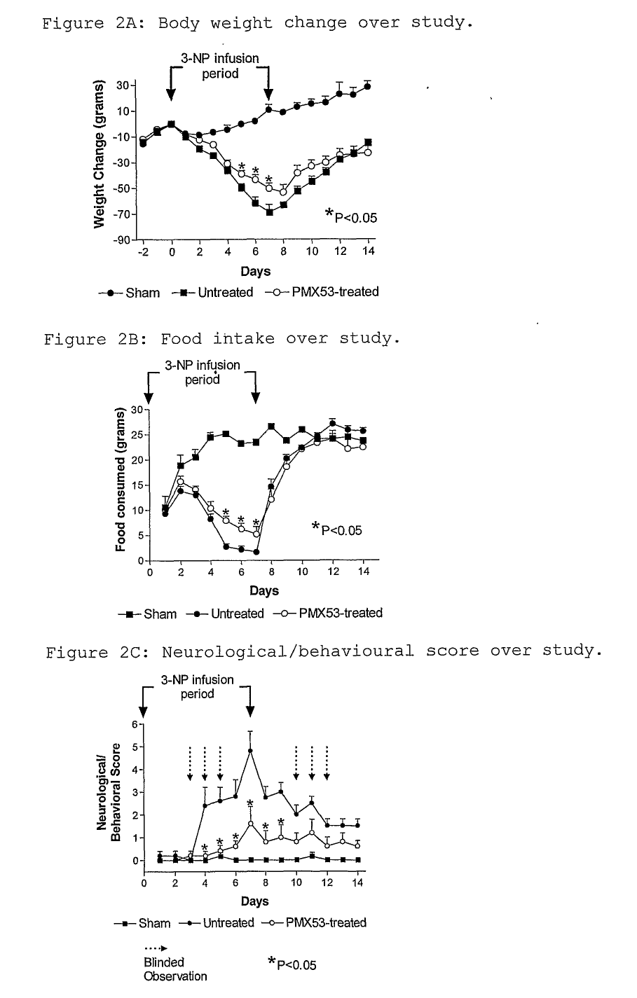

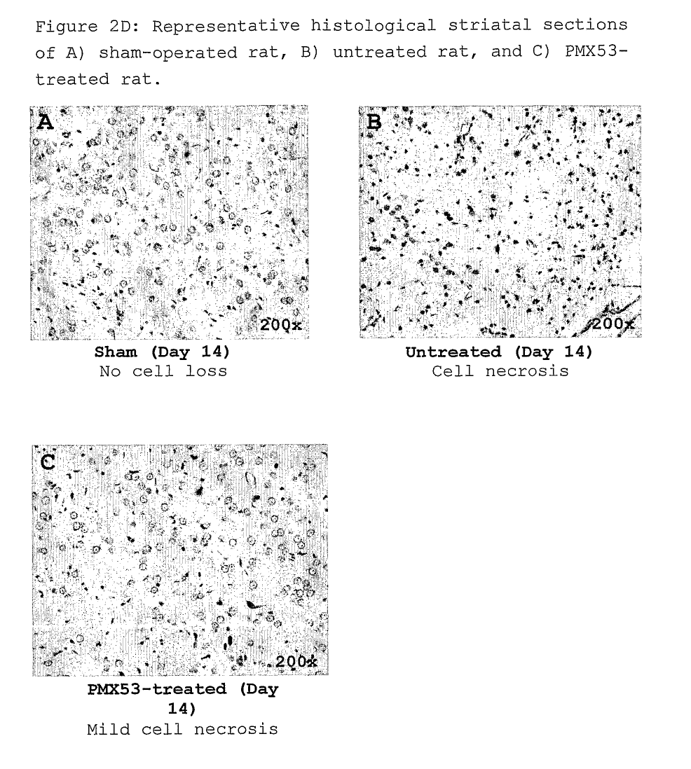

The results are shown in FIGS. 2A-2D. These show that both the untreated and PMX53-treated rats showed a decline in body weight and food consumption followed by recovery, whereas the sham-operated rats showed continued growth over the period of the experiment (FIGS. 2A and 2B). PMX53-treated rats had significantly less loss of body weight on Days 5-7 (P<0.05, n=5; FIG. 2A), as well as significantly greater food intake on Days 5-7 (P<0.05, n=5; FIG. 2B). The untreated rats showed a sharp increase in neurological/behavioural score over the period of 3-NP infusion commencing at Day 4 and peaking at Day 7, followed by a decrease to only slightly-elevated levels at Day 12 (ie 5 days after cessation of infusion). In contrast, the PMX53-treated rats showed only a slight increase in score, again with a peak at Day 7 followed by a decrease to only slightly-elevated levels at Day 12. PMX53-treated rats had significantly decreased neurological/behavioural scores on Days 4-9 (P<0.05, n=5; FIG. 2C). The sham-treated rats showed no significant change in score over the period of the experiment.

Histologically, the striatal sections of brains from untreated rats in this preliminary study showed cell necrosis and lesions consistent with findings in previously published studies (FIG. 2D). However, rats treated with PMX53 displayed fewer necrotic cells in the striatal regions than did controls.

Because it was found that after Day 7 all the rats began to recover, it was decided to stop subsequent experiments at the period of the greatest difference between the groups, i.e. after the first 7 days, so there was no need to remove the pumps.

### EXAMPLE 2

### Comparison with Other Agents

The effect of PMX53 was compared with that of a second compound of Formula I, PMX205 (Hydrocinnamate-[OpdChaWR] (HC-[OPdChaWR]), and of the known anti-inflammatory agents ibuprofen and infliximab. The groups of rats and numbers in each group were as follows:

| | |
|---|---|
| Sham | 4 |
| Untreated | 6 |
| PMX53 | 4 |
| PMX205 | 4 |
| Ibuprofen | 5 |
| Infliximab | 4 |

PMX53 (10 mg/kg/day) and PMX205 (10 mg/kg/day) were administered daily by gavage and ibuprofen (30 mg/kg/day) administered in the drinking water, beginning 2 days prior to 3-NP administration. Infliximab was administered as a single 5 mg/kg i.v. infusion on Day 0.

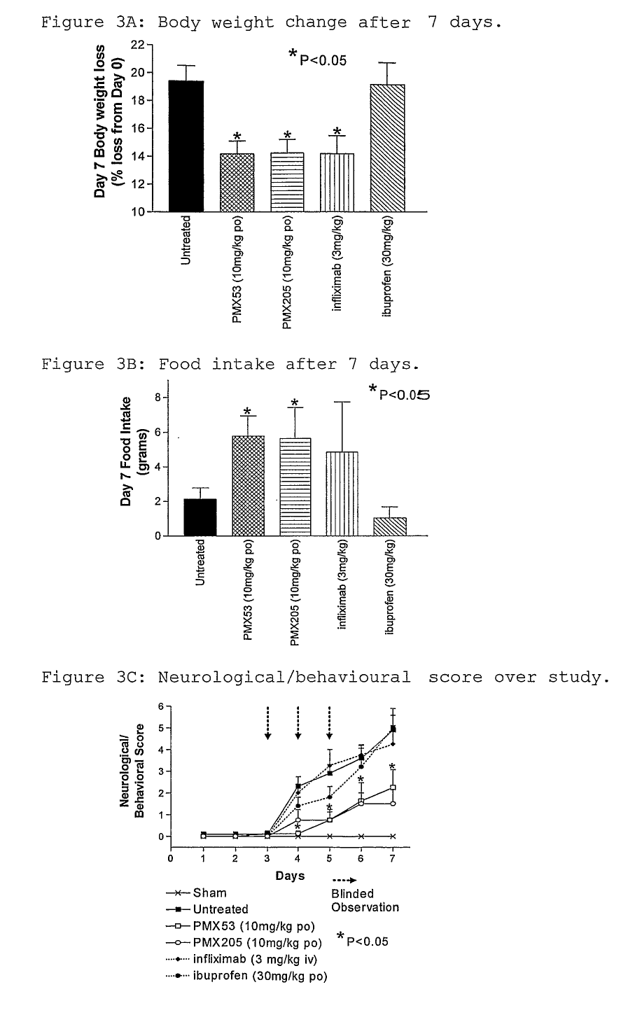

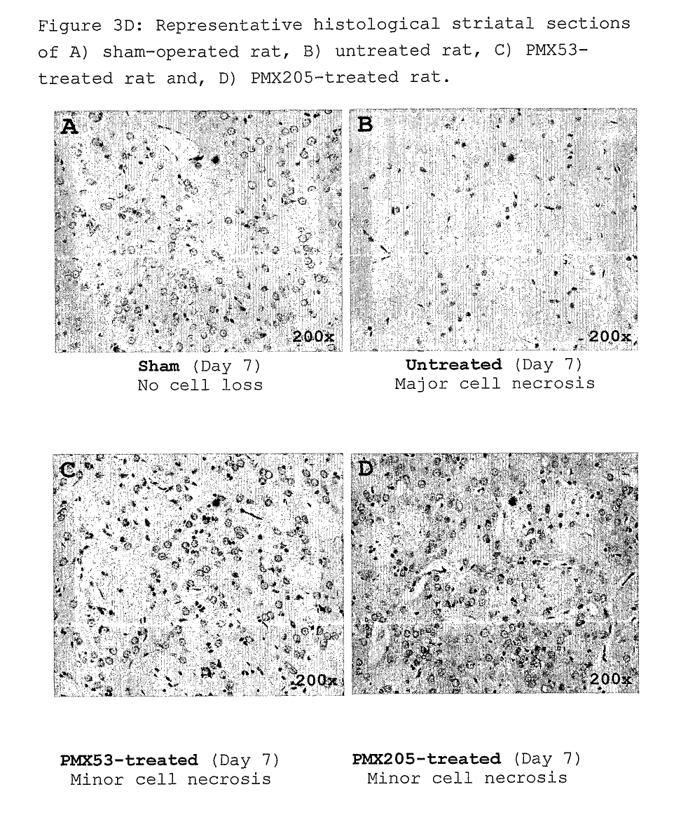

The results are shown in FIGS. 3A-3D. FIGS. 2A and 2B show that the degree of weight gain and food consumption after 7 days were similar to those observed in Example 1. For the neurological/behavioural score, both PMX53 and PMX205 provided significant protection against the adverse effects of 3-NP, while infliximab showed little if any effect, and ibuprofen showed an effect only up to Day 5 (FIG. 3C).

As shown in FIG. 3D, histological examination of sections of the striatal regions of brains from untreated rats showed marked cell necrosis and visible macroscopic lesions of a greater degree than that observed in Example 1. Rats treated with PMX53 displayed fewer necrotic cells in the striatal regions, which were similar to sections of PMX53-treated rats in Example 1.

This experiment was extended to include larger numbers of animals, and a further compound of the invention, PMX201. In addition the size of the lesions in the brain was calculated by examining pictures of brain sections stained with Nissl stain and calculating the lesion size using computerized software analysis. The groups of animals were as follows:

| | |
|---|---|
| Untreated | 26 |
| PMX53 | 21 |
| PMX205 | 19 |
| PMX201 | 3 |
| infliximab | 10 |
| ibuprofen | 12 |

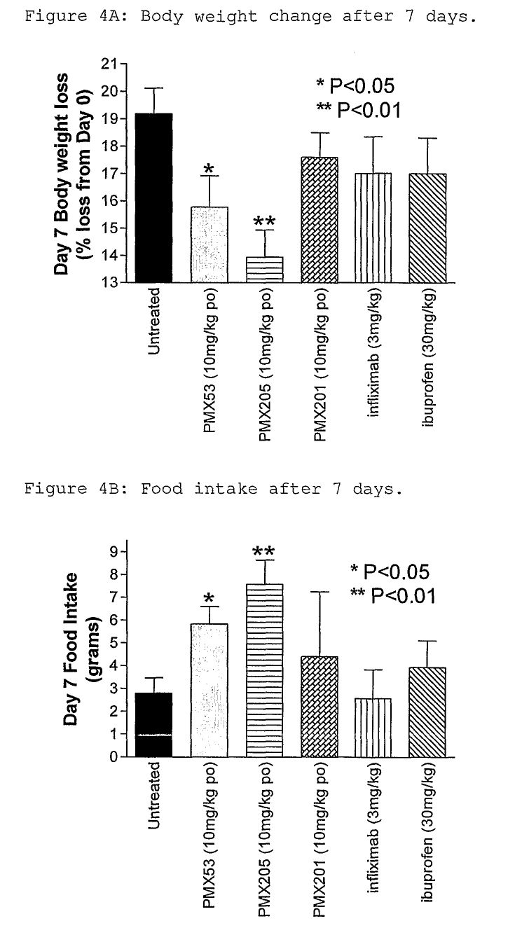

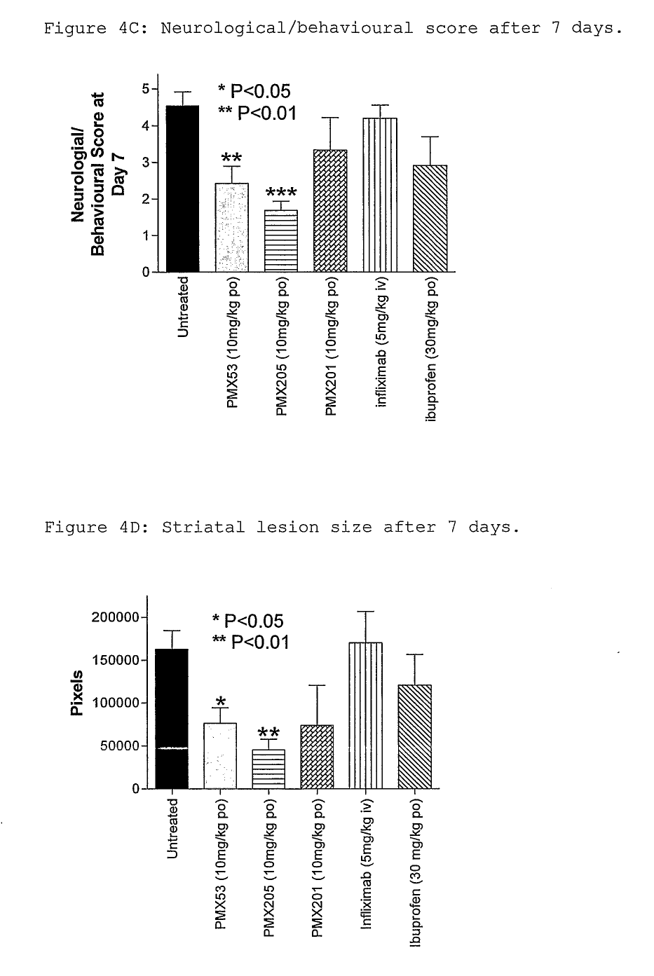

The results are summarized in FIG. 4, and show that all three PMX compounds tested ameliorated the adverse effects of 3-NP. The effect of both PMX53 and PMX205 was statistically significant for all four parameters tested; however the numbers of animals treated with PMX201 were limited by availability of compound, and were too small to assess significance. This part of the experiment is being repeated as more compound PMX201 becomes available.

### EXAMPLE 3

### Effect of Analogues of PMX53

The following compounds of Formula I are tested in the same way as described in Example 2:

### PMX205: HC-[OPdChaWR]

### PMX273: AcF-[OPdPheWR]

### PMX201: AcF-[OPdChaWCitrulline]

### PMX218: HC-[OPdPheWR]

All drugs are administered by gavage at a dose of 10 mg/kg/day, starting on day -2. If this dose is found to be effective, doses of 3 and 1 mg/kg/day or less are also tested in order to determine the dose-response relationship. The dose-response relationship for PMX53 is also determined.

The effects of these agents are also compared with those of infliximab (5 mg/kg single i.v. injection on Day 0) and ibuprofen (30 mg/kg) in drinking water.

### EXAMPLE 4

### Histological and Histochemical Examination of Samples from Example 2

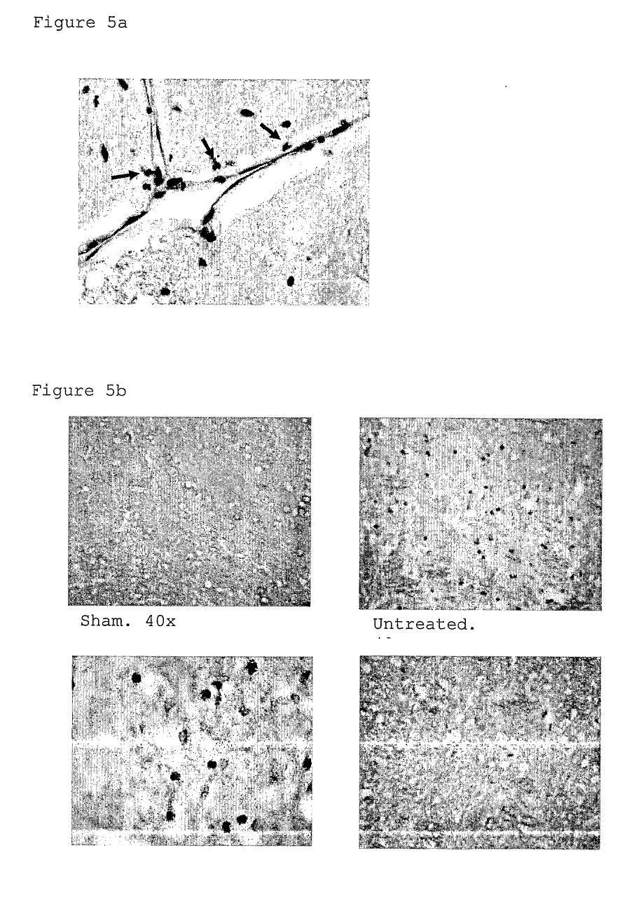

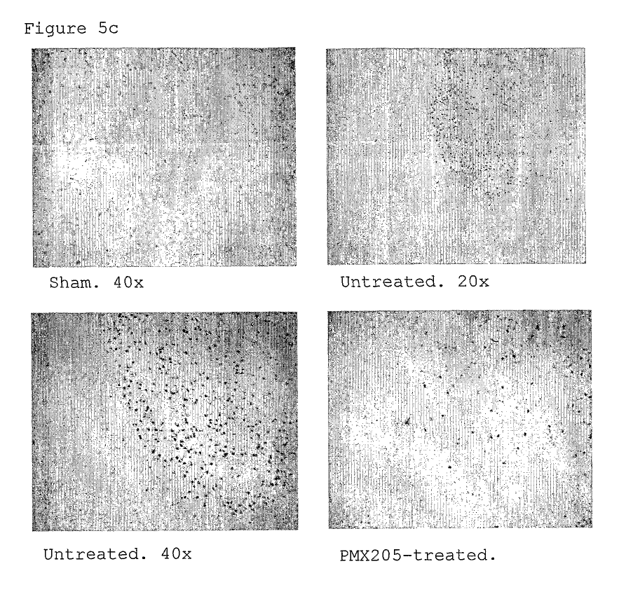

Paraffin sections of brain tissue from the rats used in Example 2 were stained with hematoxylin and eosin and examined under the microscope. Extensive infiltration of inflammatory cells was evident around and inside lesions, as shown in FIG. 5a, and this was confirmed by specific staining for neutrophils with naphthyl esterase stain as illustrated in FIG. 5b.

The TUNEL (TdT-mediated dUTP Nick-End Labeling) assay (Gavrieli, Y., et al. (1992) J. Cell. Biol. 119, 493.) measures nuclear DNA fragmentation, which is an important biochemical hallmark of apoptosis. This method has been used to demonstrate apoptosis of motor neuron s in Werdnig-Hoffmann disease, a form of spinal muscular atrophy (Simic et al, 2000). Brain sections were therefore also stained for TUNEL analysis using a standard kit (ApopTag Plus/TUNEL method; Chemicon). Striatal sections of brains containing the area of interest were fixed to a slide and deparaffinzed. Slides were then pretreated with Proteinase K (20 μg/mL), and endogenous peroxidases quenched with 3% hydrogen peroxide. Equilibration buffer was then added, followed by TdT enzyme for 1 hour. Samples were then washed, and anti-digoxigenin conjugate added for 30 min. Peroxidase substrate (diaminobenzidine) was then added, and colour allowed to develop over 6 min. Samples were then counterstained with 0.5% methyl green and mounted with Permount. Sections were washed in PBS (0.1 M, ph 7.4) in between each step. FIG. 5c shows that the lesions clearly contained apoptotic cells.

In all cases, treatment with the C5a receptor antagonists PMX53 and PMX205, completely prevented neutrophil infiltration and reduced the degree of apoptosis.

### EXAMPLE 5

### Immunohistochemical Analysis

Sections of rat brain from Example 2 were also stained with specific antibodies directed against various complement components. A standard kit (IHC Select; Chemicon) was used to stain sections for C3, C9 and C5a receptor.

For C5a receptor (C5aR) staining, the primary monoclonal antibody, mouse anti-rat C5aR was purchased from HyCult Biotechnology. This antibody has been shown to be specific for rat C5a receptors (Rothermel et al., 2000). Brain striatal sections containing the area of interest were fixed to a slide and deparaffinzed. Slides were treated with citrate buffer (pH 6.0) for 40 min at 80° C. to unmask antigens. Slides were then blocked with serum, followed by a 1:100 dilution of the primary antibody and incubated for 2 hours. Endogenous peroxidases were then quenched with 3% hydrogen peroxide, and secondary antibody (rabbit anti-mouse IgG) added for 2 hours. Sections were then incubated with streptavidin-horse radish peroxidase and then incubated with diaminobenzidine for 6 min, or until colour developed. Sections were then mounted with Permount. All sections were washed in PBS between each step.

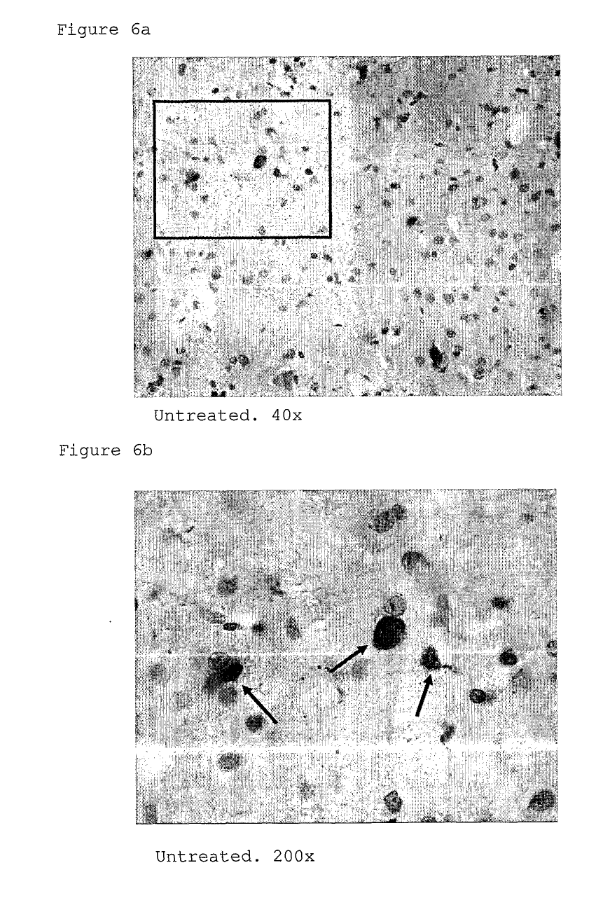

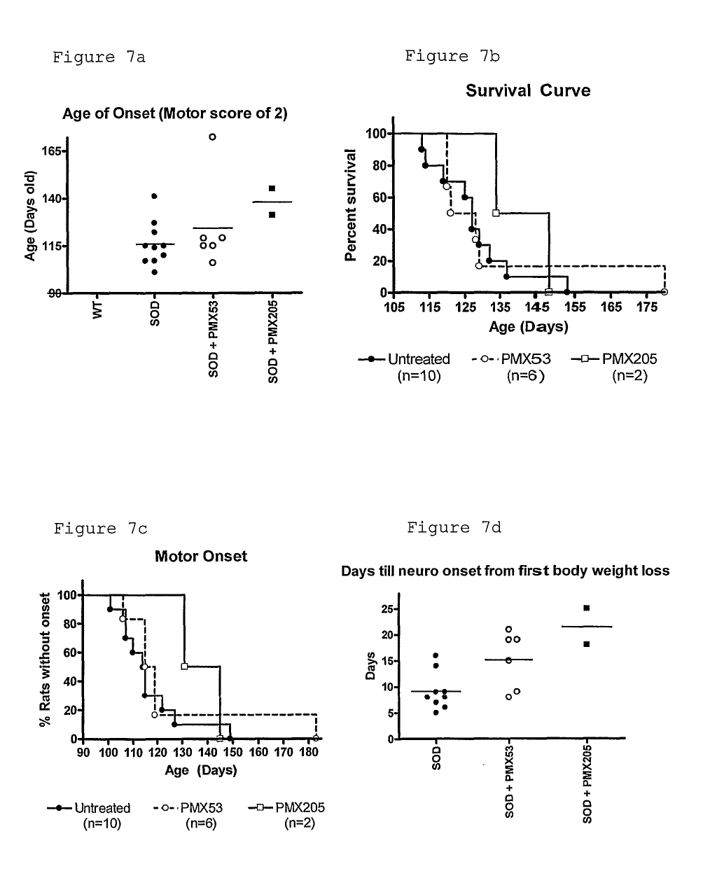

For C3 staining, rabbit anti-rat C3 antibody was purchased from Bethyl Laboratories. This polyclonal antibody has been characterized to some extent (Julian et al., 1992). For C9 staining, rabbit anti-rat C9 antiserum was obtained from Prof BP. Morgan (University of Wales College of Medicine, Cardiff). This antibody has been well characterized (Linington et al., 1989). These antibodies were used as described above, except that the antibodies were incubated at a 1:10 concentration and goat anti-rabbit IgG secondary antibodies were used. A representative result is illustrated in FIG. 6, which shows staining for C5a receptor. The dark-stained cells are cells, probably activated microglia, which are expressing C5a receptors, and are found around the edges of the lesions. Sham-treated animals showed no detectable staining.

Strong upregulation of complement components C3 and C9 and of C5a receptors was also observed around the edges of the lesions. This indicates that complement activation and concomitant increased C5a receptor expression is a critical process in the pathology of this model, and thus explains the marked therapeutic effects seen with the C5a receptor antagonists of the invention. The marked upregulation of complement in the brains of these rats following an indistinct trauma, namely mitochondrial ischemia, which leads to cell death, suggests that complement activation and upregulation in the brain may be a common pathway which operates in various kinds of brain trauma, such as stroke, trauma and neurodegenerative conditions.

### EXAMPLE 6

### Further Studies in the 3-NP Model

It is postulated that complement C5a binds to upregulated C5a receptors on brain cells (neurons and glia), and promotes inflammatory cell infiltration, and eventual lesion formation (brain cell necrosis/apoptosis). Experiments are currently in progress to examine rats at various time-points throughout the 7 days used in this model, in order to see if complement activation in the brain occurs before visible lesions can be detected.

Other studies are under way to ascertain whether the C5a antagonists are able to reverse pathology. We are currently dosing rats with PMX205 (10 mg/kg/day po) from 2 days after the commencement of 3-NP administration.

Striatal cell cultures or brain slices from the striatum are incubated in vitro in the presence of 3-NP to induce cell damage. C5a antagonists are then added to the cultures to assess their ability to prevent this damage. It is expected that this will be useful as a preliminary screening assay for selection of candidate agents for further testing in vivo.

### EXAMPLE 7

### Effect of C5a Receptor Antagonists in a Model for ALS

Transgenic Sprague-Dawley rats which carried one copy of the mutant SOD-1 gene (G63A) were purchased from the Howard Florey Institute for Physiology and Medicine, Melbourne, Australia. These rats spontaneously develop an acute form of motor neuron disease (MND; amyotrophic lateral sclerosis (ALS)) beginning at about 110-140 days of age, which has strong similarities to that seen in the human condition. Indeed, the G63A mutation is the same mutation seen in 10-20% of human patients with the familial form of ALS.

This provides a suitable model to study the efficacy of the C5a receptor antagonists. In the initial study, male rats were treated with either PMX53 (n=6), PMX205 (n=2), or with water alone for the untreated disease control group (n=10). Treatment commenced when the rats were 70 days of age. Drugs were dosed in the drinking water at approximately 1 mg/kg/day. A group of 3 wild type (WT) Sprague-Dawley rats (G63) were included as sham control animals. Rats were then monitored daily for signs of motor disturbance, using the following scale:

### Hind Limbs (score for left and right):

### No abnormality

### Noticeable muscle weakness (splaying or shaking when held by tail)

### Extreme muscle weakness (inability to dorsifex).

### Limb paralysis

### Gait:

### No abnormalities

### Abnormal gait, waddling etc.

### Righting Reflex (Time to right itself when placed on back):

0 sec (unable to successfully place on back).
<1 sec (or rat not immediately righting itself).
<5 sec
5-10 sec
>10 sec --> euthanise at this point

Body weights were also recorded daily. Rats were euthanised when they reached a score of 4 for the righting response, or had lost >20% of their peak body weight.

Following euthanasia each rat was perfused with saline (100 mL) and then formaldehyde to fix the tissues. The brain, spinal cord and the hind gastrocnemius muscles from both legs were then excised, and samples taken for histological, histochemical and electron microscopic examination.

The results are shown in FIG. 7. As illustrated in FIG. 7A, untreated rats without treatment began showing signs of loss of motor function from 101 days old, with an average onset at 116±4 days. Rats treated with PMX53 had an average age of onset of 124±10 days, while PMX205-treated rats had an average age of onset of 138±7 days. Therefore there was a clear delay in onset for the PMX205-treated group, and to a lesser extent, for the PMX53-treated group. This experiment is being repeated with larger numbers of animals to confirm the effect seen with PMX205.

FIG. 7B shows the percent survival of rats in the different groups over time. Again, there was a clear delay of mortality in the two PMX205-treated rats. In the PMX53-treated group there was possibly a small improvement compared to untreated animals.

FIG. 7C shows the percentages of rats in the different groups showing onset of motor symptoms over time. As with the survival results shown in FIG. 7C, there is a clear delay of motor onset in the two PMX205-treated rats. In the PMX53-treated rats there is possibly a small improvement compared to untreated animals.

FIG. 7D shows that in the C5a antagonist-treated rats there was an increase in the period between the first loss of body weight and the first observable loss of motor function. The results were particularly striking in the PMX205-treated rats. This suggests that the extension of survival in the drug-treated rats may result from a delay in the disease process.

These results indicate that the C5a receptor antagonists have a therapeutic effect in this transgenic rat model of MND. In particular the data suggest that PMX205 has greater efficacy and/or potency than PMX53. This is in line with trends observed with these two compounds in other disease models. A large batch of PMX205 has therefore been prepared for further studies in this model.

### EXAMPLE 8

### Studies on Tissues from Transgenic Rats Treated with C5a Receptor Antagonists

Histological analysis is currently being performed on the spinal cord and brain samples from Example 7 to estimate the number of motor neurons in the spinal cord and motor cortex, in order to determine whether the C5a receptor antagonists are reducing the death of motor neurons and hence prolonging survival. Hind limb gastrocnemius muscles are being examined for signs of end-plate degeneration using electron microscopy techniques, in order to determine whether the C5a antagonists are protecting the muscle itself from degeneration. Excised spinal cords and motor cortex are immunochemically stained with antibodies directed against different complement components, including C5a receptors, as described in Example 5, in order to elucidate whether complement upregulation is involved in the pathogenesis of this disease.

### EXAMPLE 9

### Effect of Time of Treatment with PMX205

In Example 7 we showed that a beneficial effect was obtained when the C5a receptor antagonists are administered about 1-2 months before the expected onset of disease symptoms. A group of male SOD-1 rats (n=12) is being dosed with PMX205 (1 mg/kg/day in the drinking water) from 28 days of age, in order to test whether earlier treatment gives an improved therapeutic response.

The effect of dosing rats at various stages after onset of symptoms is examined, in order to see if drug therapy can reverse active disease.

Female rats are also examined.

### EXAMPLE 10

### Do PMX Compounds Cross the Blood-Brain Barrier?

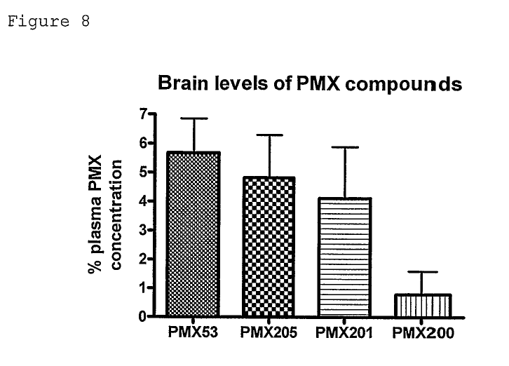

To ascertain whether PMX compounds are able to cross the blood-brain barrier, female Wistar rats (250-300 g) were anaesthetised and then 3 mg/kg of PMX53 (AcF-[OPdChaWR]), PMX205 (HC-[OPdChaWR]), PMX201 (AcF-[OPdChaWCit]) or PMX200 (HC-[OPdChaWCit]) were injected intravenously via the femoral vein. Rats were then left for 15 min, at which point a sample of blood was taken from the tail for plasma collection, and rats were then perfused via cardiac puncture with 150 mL of saline to remove blood from the brain. The brain was then dissected out. Plasma and brain samples of were prepared for pharmacokinetic analysis, and the levels of PMX compounds determined in each sample. The results, expressed as the levels determined in the brain as a percentage of the levels in the blood, are shown in FIG. 8.

All rats treated with the PMX compounds showed a degree of absorption into the brain, 15 min following i.v. administration. Rats dosed with either PMX53, PMX205 or PMX201 showed a similar level of absorption (˜7%), whereas PMX200 showed a lower degree of absorption.

These results indicate that the C5a receptor antagonists are able to cross the blood-brain barrier following systemic administration, and that removing the positive charge on the terminal arginine of the compound, via substitution with citrulline, does not appear to affect absorption. Moreover increasing the lipophilicity of the PMX compounds via substitution with either hydrocinnamate or citrulline also does not change absorption. This, together with the consistent and relatively high uptake of the PMX compounds, with the exception of PMX200, possibly indicates a specific transporter mechanism for crossing the blood brain barrier.

The absorption of the PMX compounds across the blood-brain barrier is further explored by preparing a detailed pharmacokinetic profile of absorption of the compounds to the brain following administration via various routes (i.v., i.p., s.c., p.o etc.). This includes sampling at various time points following administration. Samples of cerebrospinal fluid are also taken at various times after administration of the compound. The accumulation of PMX compounds into the brain is also examined by chronically dosing rats before sampling the brain.

### EXAMPLE 11

### Tests in Additional Cellular and Animal Models

As stated above, for further investigation of the effect of compounds of the invention on Huntington's disease a cellular model in Neuro2a cells for in vitro studies and transgenic mouse models for in vivo are available. Mouse models are also known for Kennedy disease (spinal and bulbar muscular atrophy) and for spinocerebellar ataxia-1.

Compounds of Formula I may be subjected to initial screening in vitro using the Neuro2a cell model. Suitable doses of test compounds may readily be established using routine trial and error experimentation.

Compounds found to be effective in this model or in the 3-NP model are also tested in vivo using one or more of the transgenic mouse models.

The findings for food intake and weight loss indicate that the compounds of the invention show minimal toxicity, and PMX53 is undergoing clinical trials in rheumatoid arthritis and psoriasis. The person skilled in the art will readily be able to design appropriate clinical trial protocols to test the efficacy and safety of compounds of Formula I in the treatment of the neurological and neurodegenerative conditions listed herein.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Blum D., Gall D., Cuvelier L. and Schiffmann S. Neuropharmacol and Neurotoxicol 2001, 12 1769-1772.
Blum D., Gall D., Galas M -C., d'Alcantara P., Bantobungi K., and Schiffmann S. J. Neuroscience 2002, 22 9122-9133.
Fairlie D. P., Wong A. K., West M. W. Curr. Med. Chem., 1998, 5, 29-62.
Fairlie D. P., Abbenante G. and March D. Curr. Med. Chem., 1995 2 672-705.
Gavrieli Y., Sherman Y. and Ben-Sasson S. A. Identification of programmed cell death in situ via specific labelling of nuclear DNA. J Cell Biol 1992 119 493-501
Gerard C and Gerard N. P. Ann. Rev. Immunol., 1994 12 775-808.
Howland D. S., Liu J., She Y., Goad B., Maragakis N. J., Kim B., Erickson J., Kulik J., DeVito L., Psaltis G., DeGennaro L. J., Cleveland D. W., Rothstein J. D. Focal loss of the glutamate transporter EAAT2 in a transgenic rat model of SOD1 mutant-mediated amyotrophic lateral sclerosis (ALS). Proc Natl Acad Sci USA 2002 99:1604-1609
Julian J., Carson D. D., Glasser S. R. Polarized rat uterine epithelium in vitro: constitutive expression of estrogen-induced proteins. Endocrinology. 1992, 130(1):79-87.
Katsuno M., Adachi H., Doyu M., Minamiyama M., Sang C., Kobayashi Y., Inukai A., Sobue G Leuprorelin rescues polyglutamine-dependent phenotypes in a transgenic mouse model of spinal and bulbar muscular atrophy Nat. Med. 2003, 9 768-773
Klement I. A., Skinner P. J., Kaytor M. D., Yi H., Hersch S. M., Clark H. B., Zoghbi H. Y., and Orr H. T. Ataxin-1 Nuclear Localization and Aggregation: Role in Polyglutamine-Induced Disease in SCA1 Transgenic Mice. Cell (1998) 95: 41-53.
Konteatis Z. D., Siciliano S. J., Van Riper G., Molineaux C. J., Pandya S., Fischer P., Rosen H., Mumford R. A., and Springer M. S. J. Immunol., 1994 153 4200-4204.
La Spada A. R., Wilson E. M., Lubahn D. B., Harding A. E., Fischbeck K H. Androgen receptor gene mutations in X-linked spinal and bulbar muscular atrophy. Nature 352, 77-79 (1991).
Mangiarini L., Sathasivam K., Seller M. Exon 1 of the HD gene with an expanded CAG repeat is sufficient to cause a progressive neurological phenotype in transgenic mice. Cell 87, 493-506 (1996).
McManamny P., Chy H. S., Finkelstein D. I., Craythorn R. G., Crack P. J., Kola I., Cheema S. S., Horne M. K., Wreford N. G., O'Bryan M. K., de Kretser D. M. and Morrison J. R. A mouse model of spinal and bulbar muscular atrophy. Human Molecular Genetics 11(18):2103 (2002)
Nishino N., Hida H., Kumazaki M., Shimano Y., Nakajima K., Shimizu H., Ooiwa T. and Baba H. The striatum is the most vulnerable region in the brain to mitochondrial energy compromise: a hypothesis to explain the specific vulnerability. Journal of Neurotrauma 2000, 17 251-260.
Palfi S., Ferrante R. J., Brouillet E., Beal M. F., Dolan R. Guyot M. C., Peschanski M., and Hantraye P. Chronic 3-Nitropropionic Acid Treatment in Baboons Replicates the Cognitive and Motor Deficits of Huntington's Disease J. Neuroscience 1996, 16 3019-3025.
Ralph G. S., Radcliffe P. A., Day D. M., Carthy J. M., Leroux M. A., Lee D. C. P., Wong L. F., Bilsland L. G., Greensmith L., Kingsman S. M., Mitrophanous K. A., Mazarakis N. D. & Azzouz M. Silencing mutant SOD1 using RNAi protects against neurodegeneration and extends survival in an ALS model Nature Medicine, Advance online publication doi:10.1038/nm1205, March 2005
Raoul C., Abbas-Terki T, Bensadoun J. C., Guillot S, Haase G., Szulc J., Henderson C. E., & Aebischer P. Lentiviral-mediated silencing of SOD1 through RNA interference retards disease onset and progression in a mouse model of ALS Nature Medicine, Advance online publication, doi:10.1038/nm1207, March 2005
Ross C. A. Polyglutamine pathogenesis: emergence of unifying mechanisms for Huntington's disease and related disorders. Neuron 2002, 35 819-822.
Rothermel E., Gotze O., Zahn S., Schlaf G. Analysis of the tissue distribution of the rat C5a receptor and inhibition of C5a-mediated effects through the use of two MoAbs. Scand J. Immunol. 2000, 52(4):401-10.
Sanderson S. D., Kirnarsky L., Sherman S. A., Vogen S. M., Prakesh O., Ember J. A., Finch A. M. and Taylor S. M. Decapeptide agonists of human C5a: the relationship between conformation and neutrophil response J. Med. Chem., 1995 38 3669-3675.
Shimano Y., Kumazaki M., Sakurai T., Hida H., Fujimoto I., Fukuda A. and Nishino N. Chronically administered 3-nitropropionic acid produces selective lesions in the striatum and reduces muscle tonus. Obesity Research 1995, 3 779S-784S
Simic G., Seso-Simic D., Lucassen P. J., Islam A., Krsnik Z., Cviko A., Jelasic D., Barisic N., Winblad B., Kostovic I., Kruslin B. Ultrastructural analysis and TUNEL demonstrate motor neuron apoptosis in Werdnig-Hoffmann disease J Neuropathol Exp Neurol 2000 59 398-407
Singhrao S. K., Neal J. W., Morgan B. P. and Gasque P. Increased Complement Biosynthesis By Microglia and Complement Activation on Neurons in Huntington's Disease. Experimental Neurology 1999, 159 362-376.
Strauss K. A. and Morton D. H. Type 1 glutaric aciduria, Part 2: A model of acute striatal necrosis. Amer. J. Medical Genetics (Semin. Med. Genet.) 2003, 121C 53-70
Tyndall J. D. A. and Fairlie D. P. Macrocycles mimic the extended peptide conformation recognized by aspartic, serine, cysteine and metallo proteases Curr. Med. Chem. 2001, 8, 893-907.
Wang G. H., Mitsui K., Kotliarova S., Yamashita A., Nagao Y., Tokuhiro S., Iwatsubo T., Kanazawa I., Nukina N. Caspase activation during apoptotic cell death induced by expanded polyglutamine in N2a cells. Neuroreport 1999 10(12), 2435-2438.
Zoghbi H. Y. and Orr H. T. Glutamine repeats and neurodegeneration Ann. Rev. Neurosci. 2000 23, 217-247.

1. A method of treatment of a neurological or neurodegenerative condition involving inflammation, comprising the step of administering an effective amount of an inhibitor of C5a receptor to a subject in need of such treatment.

2. The method of claim 1, in which the condition is one associated with increased activity of the complement pathway.

3. The method of claim 1, in which the inhibitor is a compound which
(a) is an antagonist of the C5a receptor,
(b) has substantially no agonist activity, and
(c) is a cyclic peptide or peptidomimetic compound of Formula I 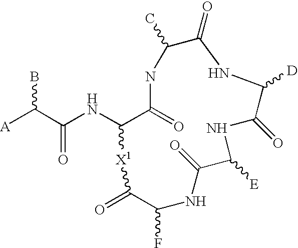
where A is H, alkyl, aryl, NH₂, NH-alkyl, N(alkyl)₂, NH-aryl, NH-acyl, NH-benzoyl, NHSO₃, NHSO₂-alkyl, NHSO₂-aryl, OH, O-alkyl, or O-aryl;
B is an alkyl, aryl, phenyl, benzyl, naphthyl or indole group, or the side chain of a D- or L-amino acid, but is not the side chain of glycine, D-phenylalanine, L-homophenylalanine, L-tryptophan, L-homotryptophan, L-tyrosine, or L-homotyrosine;
C is the side chain of a D-, L- or homo-amino acid, but is not the side chain of isoleucine, phenylalanine, or cyclohexylalanine;
D is the side chain of a neutral D-amino acid, but is not the side chain of glycine or D-alanine, a bulky planar side chain, or a bulky charged side chain;
E is a bulky substituent, but is not the side chain of D-tryptophan, L-N-methyltryptophan, L-homophenylalanine, L-2-naphthyl, L-tetrahydroisoquinoline, L-cyclohexylalanine, D-leucine, L-fluorophenylalanine, or L-histidine;
F is the side chain of L-arginine, L-homoarginine, L-citrulline, or L-canavanine, or a bioisostere thereof; and
X is —(CH₂)ₙNH— or (CH₂)ₙ—S—, where n is an integer of from 1 to 4; —(CH₂)₂O—; —(CH₂)₃O—; —(CH₂)₃—; —(CH₂)₄—; —CH₂COCHRNH—; or —CH₂—CHCOCHRNH—, where R is the side chain of any common or uncommon amino acid.

4. The method of claim 3, in which n is 2 or 3.

5. The method of claim 3, in which A is an acetamide group, an aminomethyl group, or a substituted or unsubstituted sulphonamide group.

6. The method of claim 5, in which A is a substituted sulphonamide, and the substituent is an alkyl chain of 1 to 6 carbon atoms, or a phenyl or toluoyl group.

7. The method of claim 6, in which the substituent is an alkyl chain of 1 to 4 carbon atoms.

8. The method of claim 3, in which B is the side chain of L-phenylalanine or L-phenylglycine.

9. The method of claim 3, in which C is the side chain of glycine, alanine, leucine, valine, proline, hydroxyproline, or thioproline.

10. The method of claim 3, in which D is the side chain of D-Leucine, D-homoleucine, D-cyclohexylalanine, D-homocyclohexylalanine, D-valine, D-norleucine, D-homo-norleucine, D-phenylalanine, D-tetrahydroisoquinoline, D-glutamine, D-glutamate, or D-tyrosine.

11. The method of claim 3, in which E is the side chain of an amino acid selected from the group consisting of L-phenylalanine, L-tryptophan and L-homotryptophan, or is L-1-naphthyl or L-3-benzothienyl alanine.

12. The method of claim 1, in which the inhibitor is a compound which has antagonist activity against C5aR, and has no C5a agonist activity.

13. The method of claim 1, in which the inhibitor has potent antagonist activity at sub-micromolar concentrations.

14. The method of claim 1, in which the inhibitor has a receptor affinity IC50<25 μM, and an antagonist potency IC50<1 μM.

15. The method of claim 3, in which the compound is selected from the group consisting of compounds 1 to 6, 10 to 15, 17, 19, 20, 22, 25, 26, 28, 30, 31, 33 to 37, 39 to 45, 47 to 50, 52 to 58 and 60 to 70 described in PCT/AU02/01427.

16. The method of claim 14, in which the compound is PMX53 (AcF[OP-DCha-WR]), PMX205 (HC-[OPdChaWR]), PMX273 (AcF[OP-DPhe-WR]), PMX201 (AcF[OP-DCha-WCit]) or PMX218 (HC-[OPdPheWR]).

17. The method of claim 16, in which the compound is PMX205 or PMX53.

18. The method of claim 1, in which the inhibitor is able to cross the blood-brain barrier.

19. The method of claim 1, in which the condition is a neurodegenerative condition associated with striatal lesions and/or polyglutamine repeats.

20. The method of claim 19, in which the condition is selected from the group consisting of Huntington's disease, spinal arid bulbar muscular atrophy, spinocerebellar ataxia, dentatorubral pallidoluysian atrophy, striatal injury, and acute striatal necrosis associated with Type I glutaric aciduria.

21. The method of claim 1, in which the condition is a motor neuron disease.

22. The method of claim 21, in which the condition is selected from the group consisting of amyotrophic lateral sclerosis; progressive bulbar palsy; spinal muscular atrophy, including infantile and juvenile types; Kugelberg-Welander syndrome; Duchenne's paralysis; Werdnig-Hoffmann disease; and benign focal amyotrophy.

23. The method of claim 1, in which the condition is a disorder involving neurodegeneration and/or ischemic damage.

24. The method of claim 23, in which the condition is selected from the group consisting of Parkinson's disease, Alzheimer's disease, Wilson's disease, and pathologies arising as sequelae of cerebral ischaemia and other neurological disorders, including diseases associated with dysfunction of the blood-brain barrier.

25. The method of claim 1, in which the condition is a movement disorder.

26. The method of claim 25, in which the condition is selected from the group consisting of progressive supranuclear palsy, Huntington's disease, multiple system atrophy, corticobasal degeneration, Wilson's disease, Hallervorden-Spatz disease (neurodegeneration with brain iron accumulation), progressive pallidal atrophy, Dopa-responsive dystonia-Parkinsonism, spasticity, Alzheimer's disease and other disorders of the basal ganglia which result in abnormal movement or posture.

27. The method of claim 1, in which the inhibitor is used in conjunction with one or more other agents for the treatment of the neurological or neurodegenerative condition.

28. The method of claim 27, in which the other agent is infliximab or is an inhibitor of C3a.

29-31. (canceled)
